(19) United States
(12) Patent Application Publication
(10) Pub. No.: US 2017/0203074 A1
(43) Pub. Date: Jul. 20, 2017

(54) SONIFICATION OF BIOMETRIC DATA STATE-SONGS GENERATION, BIOLOGICAL STIMULATION MODELLING AND ARTIFICIAL INTELLIGENCE

(71) Applicant: Robert Mitchell JOSEPH, Petaluma, CA (US)

(72) Inventor: Robert Mitchell JOSEPH, Petaluma, CA (US)

(21) Appl. No.: 15/405,744

(22) Filed: Jan. 13, 2017

Related U.S. Application Data
(60) Provisional application No. 62/388,076, filed on Jan. 15, 2016.

(51) Int. Cl. A61M 21/00; A61B 5/0482; G06F 19/00

(52) U.S. Cl. CPC A61M 21/00; G06F 19/345; A61B 5/0482; A61M 2021/0027

(57) ABSTRACT

A method for controlling state dependent behaviors of a user includes obtaining biometric data from a user. The method involves converting at least some of the biometric data into lines of sounds. The method involves compiling at least some of sound into a composition or song arranged to represent a targeted state. The method involves feeding the composition or song back to the user to induce the user to the targeted state.

### CROSS-REFERENCE TO RELATED APPLICATIONS

This application claims the benefit of, and priority to, U.S. Provisional Patent Application Ser. No. 62/388,076, entitled “SONIFICATION OF BIOMETRIC DATA STATE-SONGS GENERATION, BIOLOGICAL STIMULATION MODELLING AND ARTIFICIAL INTELLIGENCE,” and filed on Jan. 15, 2016, which is hereby incorporated herein in its entirety by this reference.

### BACKGROUND

An individual's “states” (i.e., happy, depressed, fearful) embody “state dependent behaviors” (smiling, inactivity, lashing out). By examples: persons suffering from PTSD are suspended in a state of trauma, and within that traumatized state, a set of negatively reactive behaviors are expressed. Outside of the traumatized state, the set of PTSD behaviors are less accessible. More positively, behaviors related to a calm state generally exclude the actions associated with trauma, stress and anxiety. Ideally, one would be able control his/her emotions so as to achieve positive states and sustain constructive behaviors. Unfortunately, the control of one's states can be elusive.

There is thus a need for systems and methods for more effectively controlling state dependent behaviors of individuals.

### BRIEF DESCRIPTION OF THE DRAWINGS

These and other features, aspects, and advantages of the present disclosure will become better understood regarding the following description, appended claims, and accompanying drawings.

FIGS. 1-3 illustrate different external and internal states for context.

FIG. 4 illustrates a method for controlling state dependent behaviors of a user according to an embodiment.

FIG. 5 illustrates a system for controlling state dependent behaviors of a user according to an embodiment

FIG. 6 illustrates a somatic module according to an embodiment.

FIG. 7 illustrates an endocrine module according to another embodiment.

FIG. 8 illustrates an EEG module according to another embodiment.

FIG. 9 illustrates a system of module integration according to an embodiment.

FIG. 10 illustrates a modelling system according to an embodiment.

FIG. 11 illustrates a modelling system according to another embodiment.

FIG. 12 illustrates a user interface according to an embodiment.

FIG. 13 illustrates a user interface according to another embodiment.

FIG. 14 illustrates a user interface according to another embodiment.

### DETAILED DESCRIPTION OF VARIOUS EMBODIMENTS

A better understanding of different embodiments of the disclosure may be had from the following description read with the accompanying drawings.

While the disclosure is susceptible to various modifications and alternative constructions, certain illustrative embodiments are in the drawings and are described below. It should be understood, however, there is no intention to limit the disclosure to the specific embodiments disclosed, but on the contrary, the intention covers all modifications, alternative constructions, combinations, and equivalents falling within the spirit and scope of the disclosure.

It will be understood that unless a term is expressly defined in this application to possess a described meaning, there is no intent to limit the meaning of such term, either expressly or indirectly, beyond its plain or ordinary meaning.

Any element in a claim that does not explicitly state “means for” performing a specified function, or “step for” performing a specific function is not to be interpreted as a “means” or “step” clause as specified in 35 U.S.C. §112(f).

Embodiments of the present disclosure transform biometric data from a user into sound which is fed back into the user to beneficially influence state dependent behaviors of the user. For instance, methods of the present disclosure can involve generating biometric based music to induce a desired or target internal state of clam, learning, and/or second wind in the user. Alternatively, methods described herein can involve generating biometric based music for use in a movie or television soundtrack to induce one or more desired audience emotions. In other embodiments, methods of the present disclosure can be adapted to provide an alternative mode and methodology for biological stimulation modelling. In yet other embodiments, methods of the present disclosure can be adapted to generate biometric based music and utilize the same as a predictive, diagnostic, and/or artificial intelligence tool as described in more detail below.

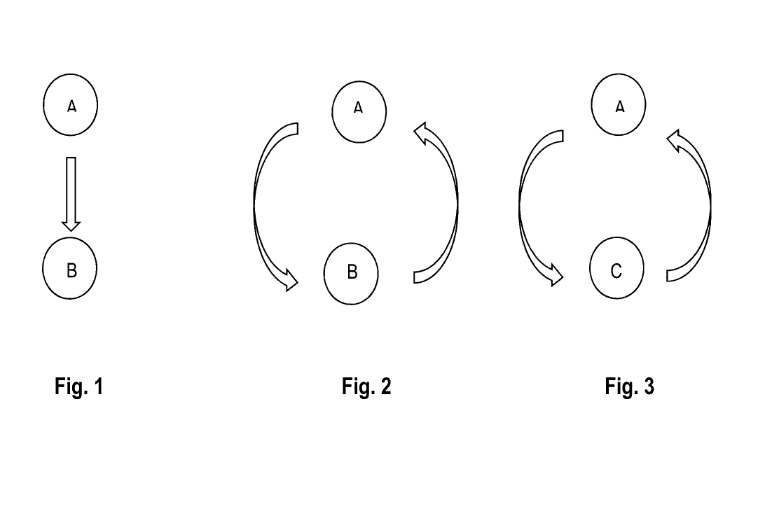

FIGS. 1-3 illustrates exemplary relationships between internal and external states of a user to provide context to the present disclosure. Referring to FIG. 1, an internal state A comprises the physiological state of depressed, and an external state B comprises a physical posture B, which when depressed, includes slumped shoulders, chin pointing down, low effect. Internal state A of depressed and the physical posture B are generally isomorphic or two different presentations of the same state at different levels, internal and external. The physical posture B is generally a result of the individual's internal state or physiology. For instance, if the internal physiology is depressed the posture physical presents as such.

Referring to FIG. 2, the relationship between the internal state A and external state or physical posture B can be reciprocal in that the physical posture B feeds back upon and impacts the internal state A in such a way that if we change the physical posture B by pulling the shoulders back, pushing the chin upwards and projecting a posture of confidence, the internal state A conforms in accordance with the physical posture B of confidence to change to an internal state of confidence. In other words, the internal state A creates the physical posture B but the physical posture B can influence or force the internal state A to conform to the physical posture B. Thus, while the external state or physical posture B is a postural isomorph derived from within the internal or physiological state A, the external state or physical posture B can control the internal state A from the outside-in. Cybernetics dictates that if the external state B is fed back into the system of the internal state A from which it was derived, the internal state A will at least in part conform to the external state B.

Referring to FIG. 3, the described relationship between the internal state A and the external state or physical posture B can be extended to include other isomorphs such as a sonic isomorph. Biometric based music can be derived from a system in an internal state A and then fed back into the system to influence the system. The biometric based music can be referred to as a state-song C. This state-song C represents a state of a system of origin and can cause the internal state A of that system to at least in part conform to the state-song C. For instance, if that state-song C is generated from biometric data obtained from a user with an elevated heart rate that user can listen to the state-song C at a different time when the user's heart rate is reduced and experience a palpable increase in its heart rate.

Systems and methods of the present disclosure can advantageously thus generate biometric based music from biometric or physiological data to create a sonic isomorph of a state that induces that same or similar state. This can be identified as a phenomenological isomorph meaning that the same result can be achieved with other state isomporphs (e.g., music). Similar to the previous example of the physiology/posture case, the state-song C derived from a specific internal state can be fed back into an user or system of origin to induce a targeted state; such as calm, aware, excitement, second wind, focus, etc.

Systems and methods of the present disclosure can be adapted to address PTSD, depression, anxiety, insomnia, and/or any other internal state so as to modify the physiology and behaviors that are encumbered by those states. Other embodiments, can be adapted to produce individualized sonic simulation models that merge somatic, EEG and endocrinological data. Alternatively, such personalized simulation models can be used by artificial intelligent agents for health diagnostics and interventions.

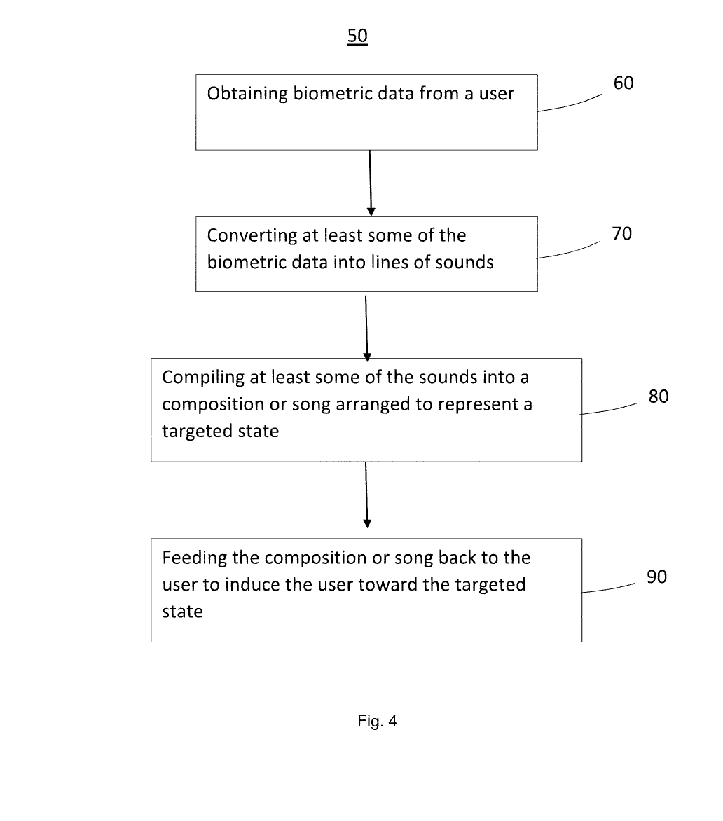

FIG. 4 illustrates exemplary steps in a method 50 for controlling state dependent behaviors of a user. In an act 60, biometric data is obtained from a user. The biometric data can include somatic data, endocrine data, cortical data, or any other suitable biometric data.

In an act 70, at least some of the biometric data is converted or transformed into lines of sound. This may be referred to as “sonifying” the biometric data. Conversion or transformation of the biometric data may include frequency/amplitude conversion and/or algorithmic processing. The conversion and/or transformation of the biometric data can be performed by one or more modules as discussed below. For instance, the biometric data can be converted or transformed into lines of sound by a processing module of a computing device.

In an act 80, at least some of the lines of sound are compiled into compositions or state-songs arranged to represent a targeted state of the user. The conversion and/or transformed of the biometric data can be performed by one or more modules as discussed below. The targeted states can include different states such as calm, learning, exercise, rest, etc.

In an act 90, the compilation or state-song is fed or provided back to the user to induce the user to the targeted state. The compilation or state-song can be fed or provided to the user via an output component such as a speaker, headphones, or any other suitable output device.

It will be appreciated that method 100 can adapted or implemented to help reduce suffering from post-traumatic stress disorder (PTSD), Anxiety, Obesity, ADHD, Schizophrenia, and/or other psychological conditions.

According a variation, the state-songs of the present disclosure can facilitate predictive analytics and personalized medicine. For instance, a user's state song can be singularly unique to that individual. From a clinical perspective this helps move health sciences past group statistics toward modelling, monitoring and intervention of a highly individualized and predictive nature. Furthermore, the parallel imbedded nature of biometrics is rich soil for deep learning. Aimed towards bio-simulations with individual accuracy, methods of the present disclosure can define the way intelligent agents learn, diagnose, predict, prevent and intervene. In addition, integration of state songs and holography may provide a platform on which to evolve and utilize a truly intelligent AI.

It will be appreciated that any of the methods described herein may be implemented in an application. The application may be software embodied on a computer readable medium which when executed by a module of a computer system performs a sequence of steps. The application may be a mobile application or application software configured to run on smartphones, tablets computers, and/or other mobile devices. The application may be a web-based programming language and/or web-based computing platform. The application may be a computer programming language that is concurrent, class-based, object-oriented, and design to have minimal implementation dependencies (e.g., Java programming language).

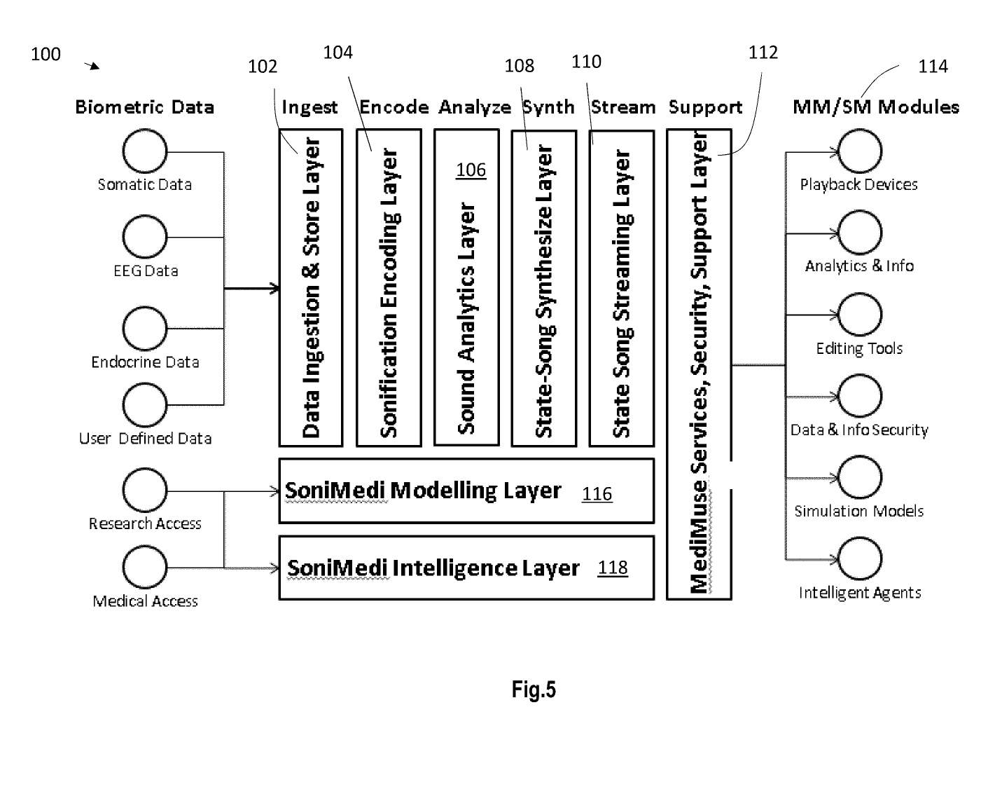

FIG. 5 schematically depicts a system 100 for controlling state dependent behavior of a user. The system 100 may include a computer device that can display information to a user and receive user input, respectively. The computer device can include a mobile device. A mobile device is defined as a processing device routinely carried by a user. It typically has a display screen with touch input and/or a keyboard, and its own power source. The system 100 may include biometric data capture devices (e.g., sensors), storage devices, and/or transmission devices.

The system 100 can be in communication with an application and/or a cloud computing platform. The application and/or cloud platform can be configured to perform any of the acts described herein. For instance, the cloud platform can be arranged to convert data (e.g., biometric data) to lines of sound, sounds and/or songs. In other embodiments, the cloud platform can be arranged to store and/or transmit data, sounds, and/or songs.

In other embodiments, the cloud platform and/or application can be arranged to manage intelligent agents and/or artificial intelligence. For instance, the cloud platform and/or application can include predictive simulation and/or health diagnostic agents. In an embodiment, the application and/or cloud platform can be arranged to perform sonificiation and/or include holographic intelligence systems.

The system 100 may include different modules 102-118 arranged to perform different functions. For instance, the system 100 may include one or more modules for biometrics data sonification, biometrics and state analysis, sound generations, state-song composition, state-song storage and access, user services and tools, biological systems modelling, and/or intelligent agents. According to a variation, the system 100 can include an ingest module 102 arranged to receive and/or store data including biometric data. The biometric data may include any suitable type of data, including, but not limited to, somatic data, EEG data, endocrine data, and/or user defined data. The system 100 can also include an encode module 104 arranged to encode or sonify data from the ingest module. The system 100 can include an MM/SM module 114 including playback devices, analytics and info, editing tools, data and info security, simulation models, and/or intelligent agents. In other embodiments, the system 100 may include input/output modules including dashboards and/or graphical user interfaces.

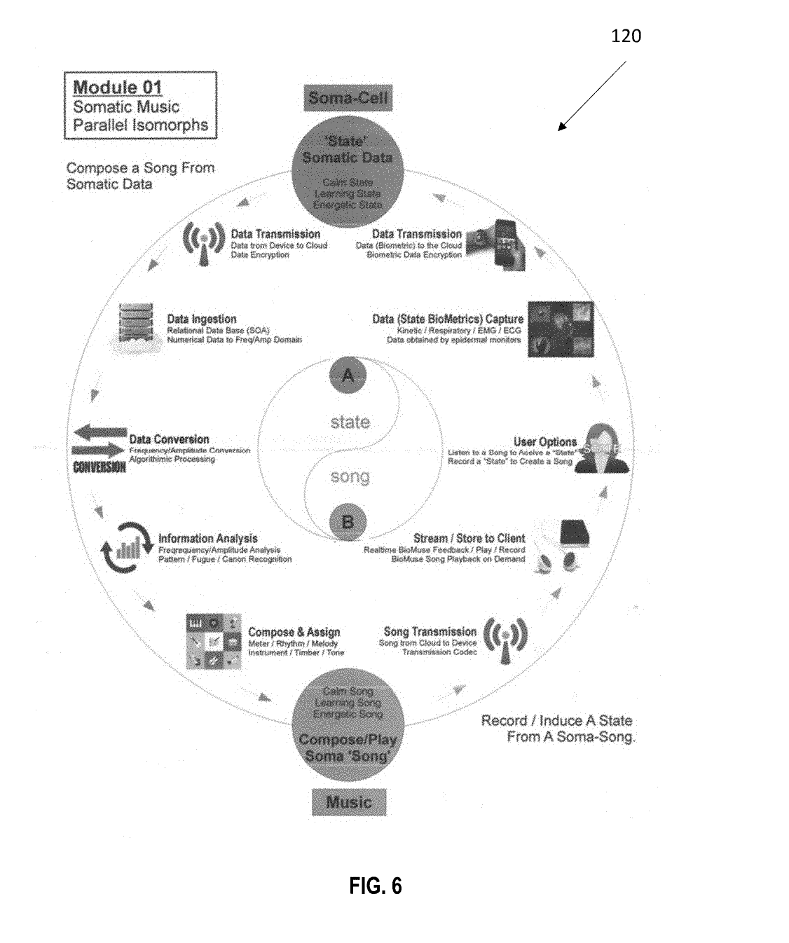

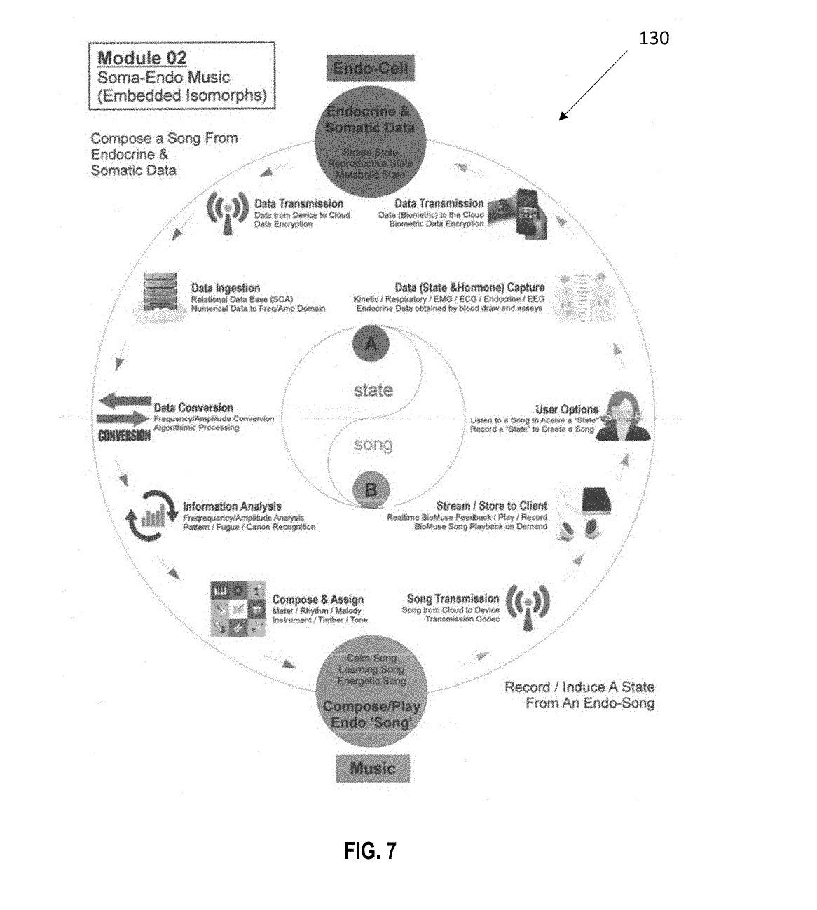

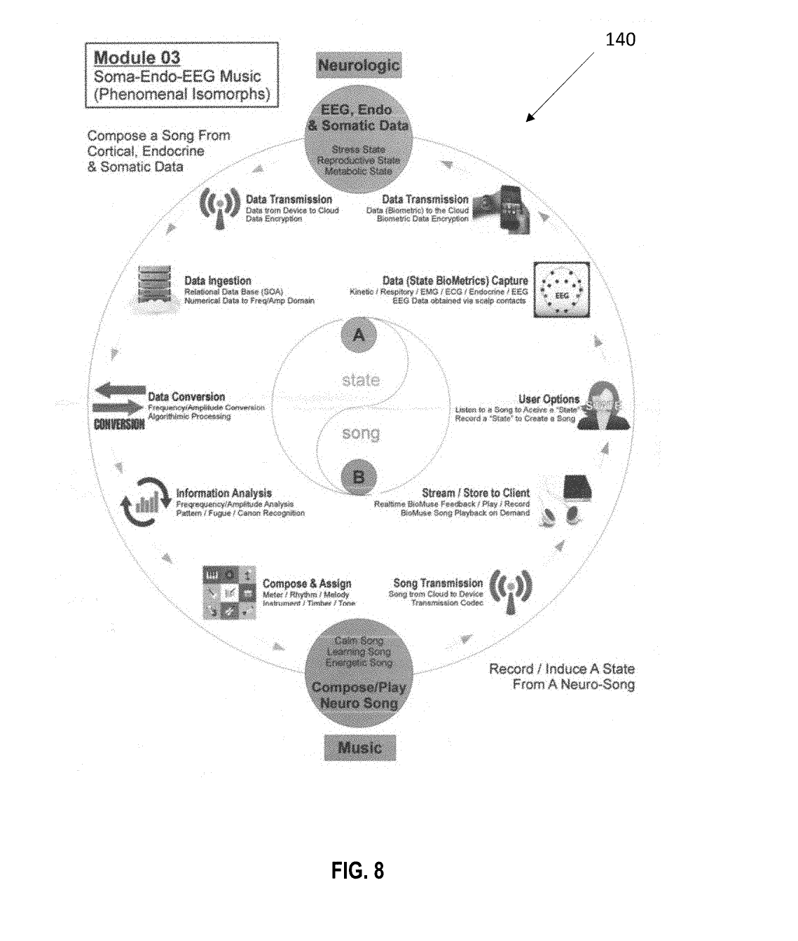

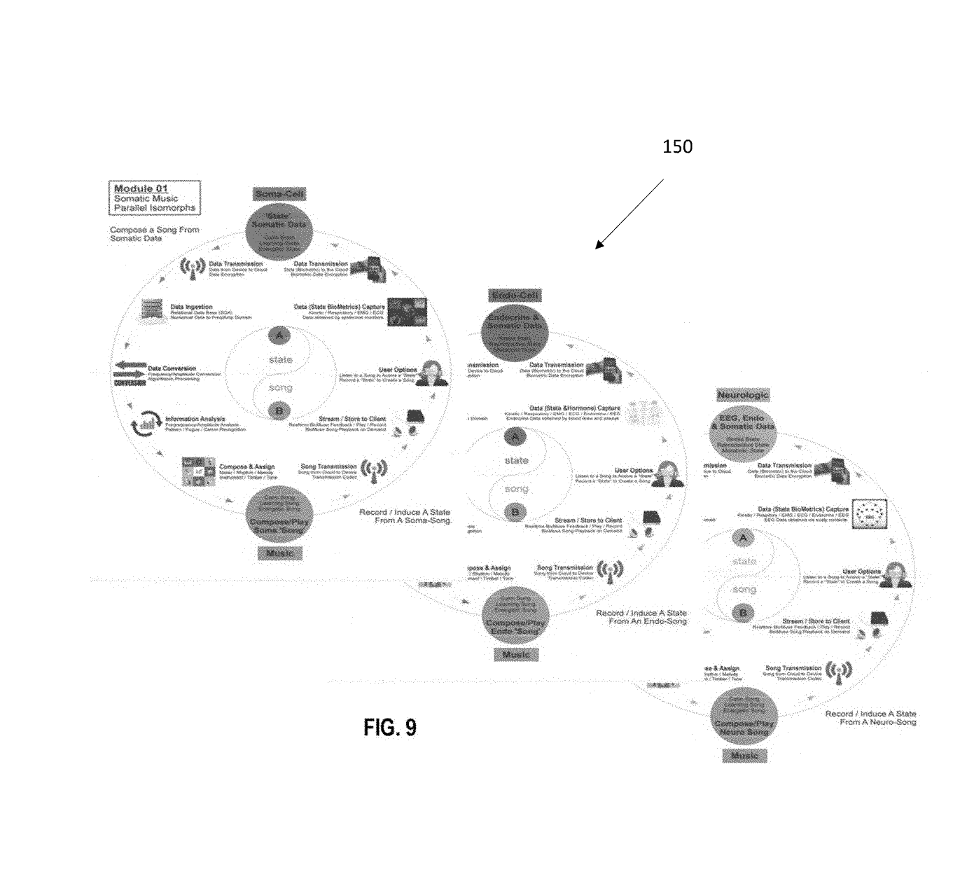

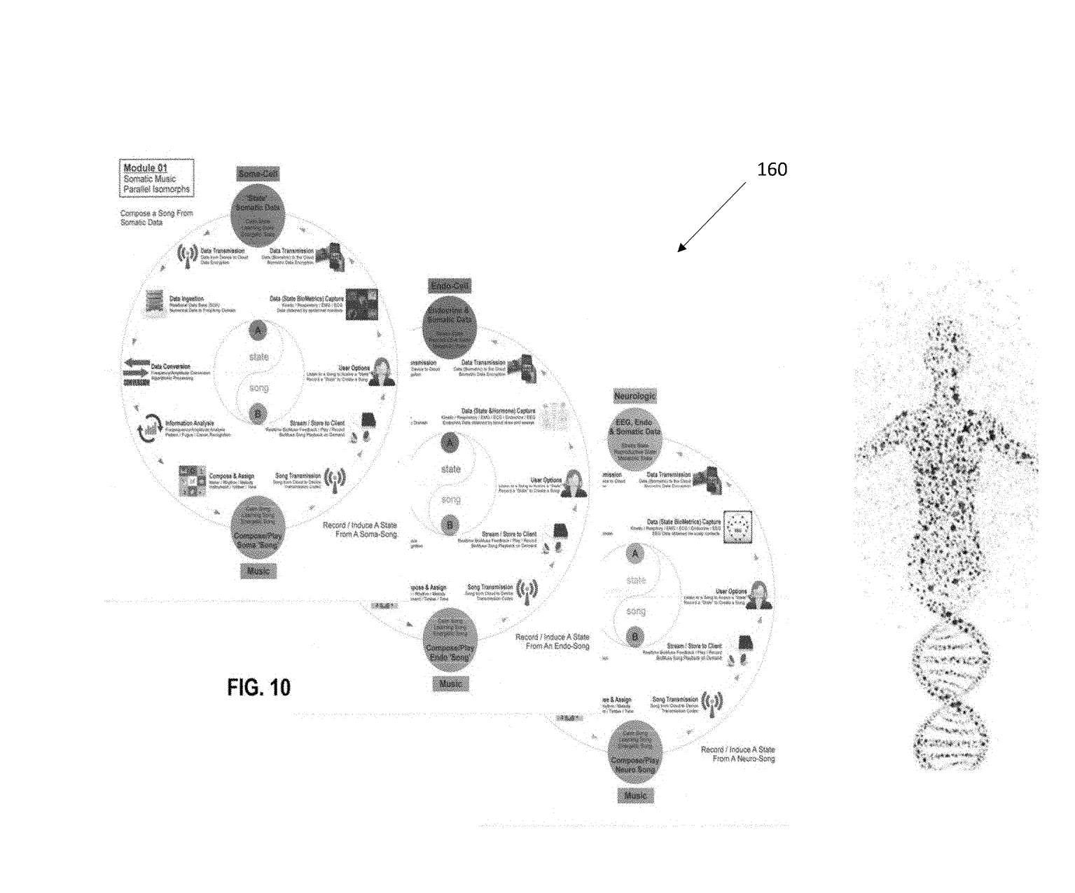

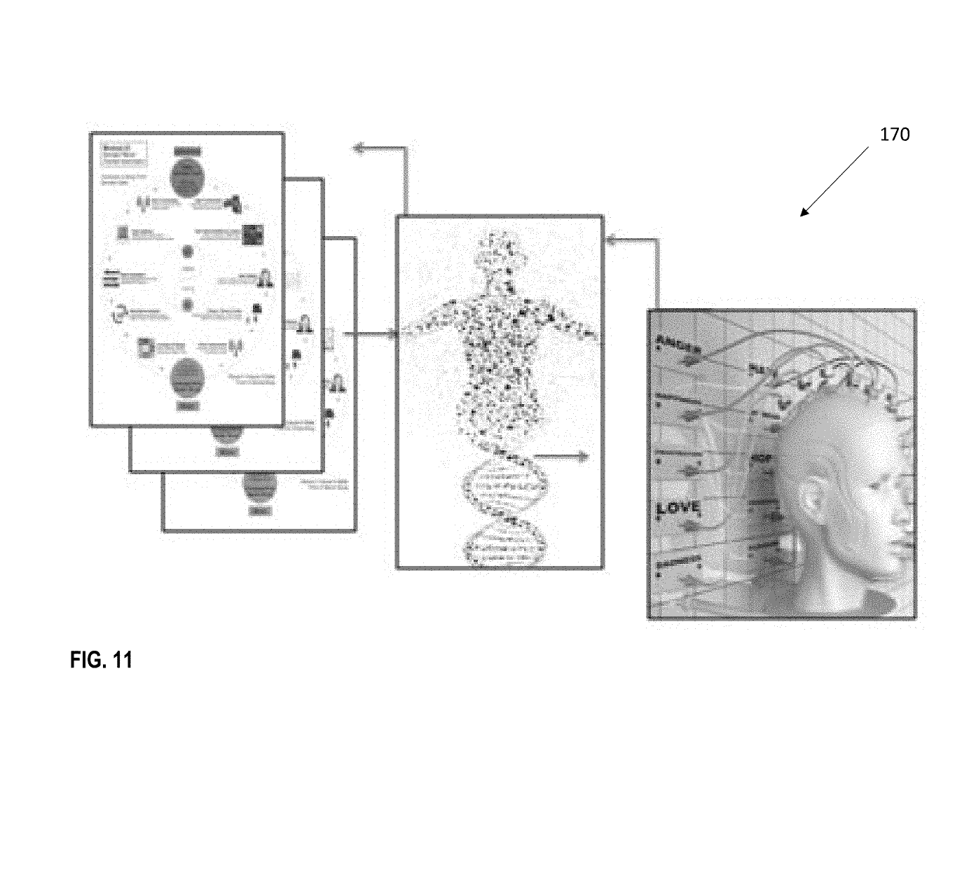

FIG. 6 illustrates a somatic module 120 according to an embodiment. FIG. 7 illustrates a somatic module 130 according to an embodiment. FIG. 8 illustrates an EEG module 140 according to an embodiment. FIG. 9 illustrates a method 150 of module integration. FIG. 10 illustrates a method 160 of biometrics and sonimodi-modelling. The method 160 includes stacking modules with data integration and comparisons towards individualized medical models. FIG. 11 illustrates a method 170 of state song, simulation modeling and AI. The method 170 includes stacking modules with data integration and medical modelling toward biological intelligence.

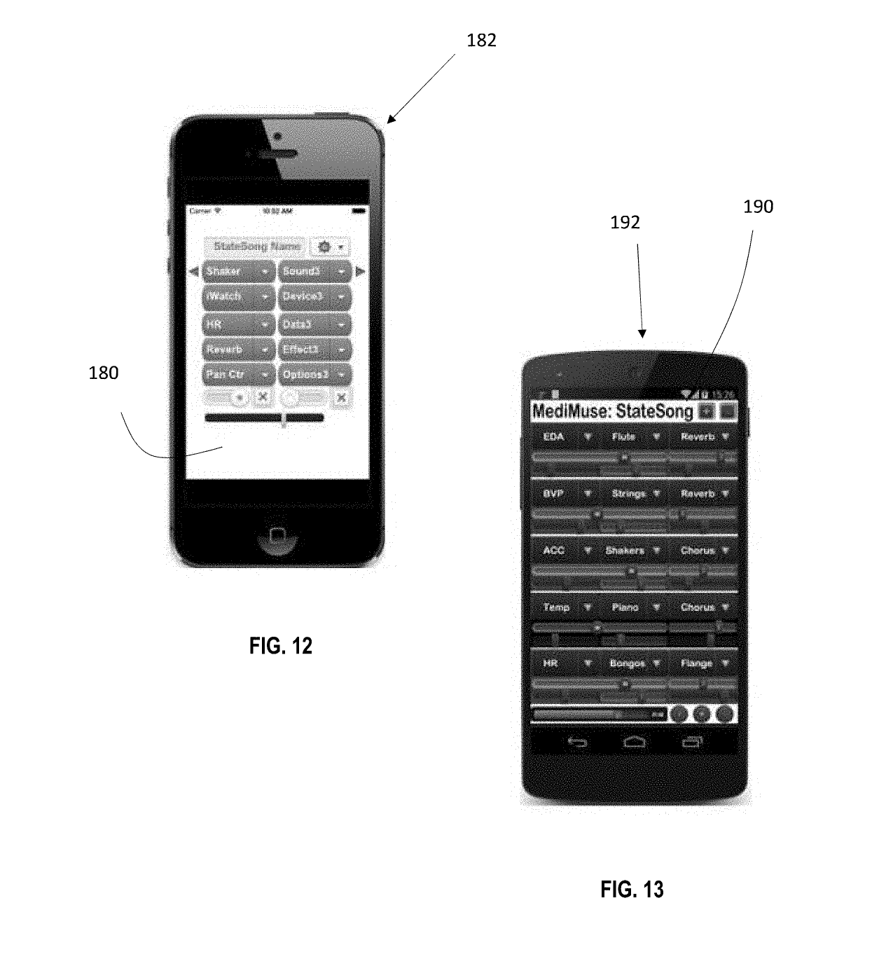

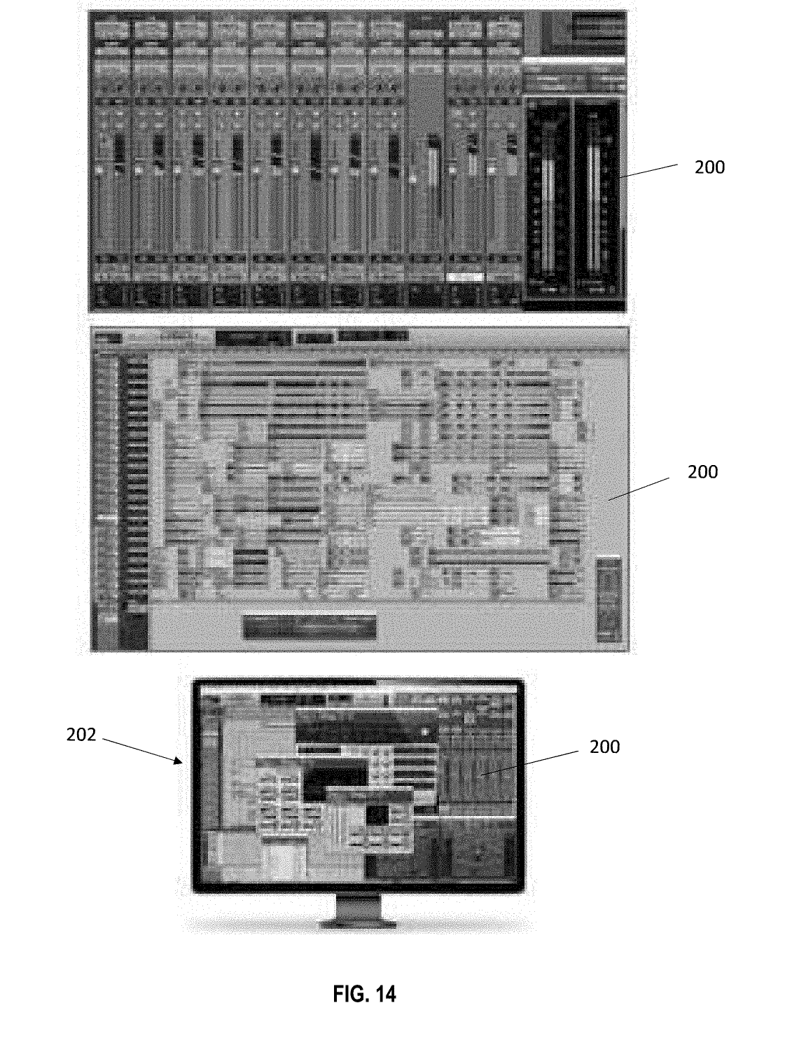

It will be appreciated that the system 100 can include one or more user interface through which the user is able to input or receive information. For instance, FIG. 12 illustrates a user interface 180 implemented on a mobile device 182. As seen, the user interface module 180 can be simplified to improve ease of use. FIG. 13 illustrates a user interface 190 implemented on a mobile device 192. The user interface 192 can include more options and controls for a more involved user. FIG. 14 illustrates a user interface 200 implemented on a desk top computer 202. The user interface 200 can have a more a complex architecture adapted for analytics and modelling.

Many of the elements described in the disclosed embodiments may be implemented as modules. A module is defined here as an isolatable element that performs a defined function and has a defined interface to other elements. The modules described in this disclosure may be implemented in hardware, a combination of hardware and software, firmware, or a combination, all of which can be behaviorally equivalent. Modules may be implemented using computer hardware in combination with software routine(s) written in a computer language. It may be possible to implement modules using physical hardware that incorporates discrete or programmable analog and/or digital hardware. Examples of programmable hardware include computers, microcontrollers, microprocessors, application-specific integrated circuits, field programmable gate arrays, and complex programmable logic devices.

As noted above, the application may be software embodied on a computer readable medium which when executed by a processor component of a computer device performs a sequence of steps. The application may be a mobile application or application software configured to run on smartphones, tablets computers, smart watches, and/or other mobile devices. Moreover, embodiments of the present disclosure may comprise or utilize a special-purpose or general-purpose computer system that includes computer hardware, such as, for example, one or more processors and system memory, as discussed in greater detail below. Embodiments within the scope of the present disclosure also include physical and other computer-readable media for carrying or storing computer-executable instructions and/or data structures. Such computer-readable media can be any available media that can be accessed by a general-purpose or special-purpose computer system. Computer-readable media that store computer-executable instructions and/or data structures are computer storage media. Computer-readable media that carry computer-executable instructions and/or data structures are transmission media. Thus, by way of example, and not limitation, embodiments of the disclosure can comprise at least two distinctly different kinds of computer-readable media: computer storage media and transmission media.

Computer storage media are physical storage media that store computer-executable instructions and/or data structures. Physical storage media include computer hardware, such as RAM, ROM, EEPROM, solid state drives (“SSDs”), flash memory, phase-change memory (“PCM”), optical disk storage, magnetic disk storage or other magnetic storage devices, or any other hardware storage device(s) which can be used to store program code in the form of computer-executable instructions or data structures, which can be accessed and executed by a general-purpose or special-purpose computer system to implement the disclosed functionality of the disclosure.

Transmission media can include a network and/or data links which can be used to carry program code in the form of computer-executable instructions or data structures, and which can be accessed by a general-purpose or special-purpose computer system. A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer system, the computer system may view the connection as transmission media. Combinations of the above should also be included within the scope of computer-readable media.

Further, upon reaching various computer system components, program code in the form of computer-executable instructions or data structures can be transferred automatically from transmission media to computer storage media (or vice versa). For example, computer-executable instructions or data structures received over a network or data link can be buffered in RAM within a network interface module (e.g., a “NIC”), and then eventually transferred to computer system RAM and/or to less volatile computer storage media at a computer system. Thus, it should be understood that computer storage media can be included in computer system components that also (or even primarily) utilize transmission media.

Computer-executable instructions comprise, for example, instructions and data which, when executed at one or more processors, cause a general-purpose computer system, special-purpose computer system, or special-purpose processing device to perform a certain function or group of functions. Computer-executable instructions may be, for example, binaries, intermediate format instructions such as assembly language, or even source code.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. As such, in a distributed system environment, a computer system may include a plurality of constituent computer systems. In a distributed system environment, program modules may be located in both local and remote memory storage devices.

Those skilled in the art will also appreciate that the disclosure may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud computing model can be composed of various characteristics, such as on-demand self-service, broad network access, resource pooling, rapid elasticity, measured service, and so forth. A cloud computing model may also come in the form of various service models such as, for example, Software as a Service (“SaaS”), Platform as a Service (“PaaS”), and Infrastructure as a Service (“IaaS”). The cloud computing model may also be deployed using different deployment models such as private cloud, community cloud, public cloud, hybrid cloud, and so forth.

Some embodiments, such as a cloud computing environment, may comprise a system that includes one or more hosts that are each capable of running one or more virtual machines. During operation, virtual machines emulate an operational computing system, supporting an operating system and perhaps one or more other applications as well. In some embodiments, each host includes a hypervisor that emulates virtual resources for the virtual machines using physical resources that are abstracted from view of the virtual machines. The hypervisor also provides proper isolation between the virtual machines. Thus, from the perspective of any given virtual machine, the hypervisor provides the illusion that the virtual machine is interfacing with a physical resource, even though the virtual machine only interfaces with the appearance (e.g., a virtual resource) of a physical resource. Examples of physical resources including processing capacity, memory, disk space, network bandwidth, media drives, and so forth.

The present disclosure may be embodied in other specific forms without departing from its spirit or essential characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

While various aspects and embodiments have been disclosed herein, other aspects and embodiments are contemplated. The various aspects and embodiments disclosed herein are for purposes of illustration and are not intended to be limiting. Additionally, the words “including,” “having,” and variants thereof (e.g., “includes” and “has”) as used herein, including the claims, shall be open ended and have the same meaning as the word “comprising” and variants thereof (e.g., “comprise” and “comprises”).

While various aspects and embodiments have been disclosed herein, other aspects and embodiments are contemplated. The various aspects and embodiments disclosed herein are for purposes of illustration and are not intended to be limiting. Additionally, the words “including,” “having,” and variants thereof (e.g., “includes” and “has”) as used herein, including the claims, shall be open ended and have the same meaning as the word “comprising” and variants thereof (e.g., “comprise” and “comprises”).

1. A method for controlling state dependent behaviors of a user:
obtaining biometric data from a user;
converting at least some of the biometric data into lines of sounds;
compiling at least some of the lines of sound into a composition or song arranged to represent a targeted state; and
feeding the composition or song back to the user to induce the user to the targeted state.

2. The method of claim 1, wherein conversion of the biometric data includes at least some frequency conversion.

3. The method of claim 1, wherein conversion of the biometric data includes at least some algorithmic processing.

4. A computer program product comprising one or more computer storage media having stored thereon computer-executable instructions that, when executed at a processor, cause the computer system to perform a method for controlling state dependent behaviors of a user, the method comprising:
an act of the computer system obtaining biometric data from the user;
an act of the computer system converting at least some of the biometric data into lines of sounds;
an act of the computer system compiling at least some of the lines of sound into a composition or song arranged to represent a targeted state; and
an act of the computer system feeding the composition or song back to the user to induce the user to the targeted state.
